(19) United States
(12) Patent Application Publication
(10) Pub. No.: US 2019/0358442 A1
(43) Pub. Date: Nov. 28, 2019

(54) Tattoo Needle Tube including a Tattoo Ink Reservoir for Manually Dispensing Tattoo Ink During Tattooing

(71) Applicants: Samuel Davenport, Shreveport, LA (US); Samuel Davenport, Shreveport, LA (US)

(72) Inventors: Samuel Davenport, Shreveport, LA (US); Samuel Davenport, Shreveport, LA (US)

(21) Appl. No.: 16/409,854

(22) Filed: May 12, 2019

Related U.S. Application Data
(60) Provisional application No. 62/670,847, filed on May 13, 2018.

(51) Int. Cl. A61M 37/00

(52) U.S. Cl. CPC A61M 37/0084

(57) ABSTRACT

The present invention teaches a cylindrical tattoo tube which includes an ink reservoir located on one side of the cylinder. A plunger may be included in the ink reservoir to control the amount of ink being dispensed. A user may manually actuate the plunger while tattooing, thereby eliminating the need constantly dip during the tattooing procedure.

### Related Applications

This application claims benefit from U.S. Provisional Application No. 62/670,847, entitled “A Tattoo Needle Tube Including a Tattoo Ink Reservoir for Manually Dispensing Tattoo Ink During Tattooing,” filed May 13, 2018.

### Field of the Invention

The present invention relates to a tattoo tube for holding a reciprocating needle for infusing insoluble ink into human or animal skin.

### BACKGROUND OF THE INVENTION

Conventional tattoo machines comprise a reciprocating needle that moves up and down to infuse insoluble ink particles into a human's or animals top layer of skin. In many cases, the needle moves linearly within a tubular or cylindrical tattoo tube or other cylindrical structure that is held in a tattoo artist hand while using the reciprocating needle to tattoo an individual. The reciprocating needle typically punctures the skin at a high rate of frequency during the tattooing process.

To facilitate tattooing, tattoo machine needles are installed in the artist's tattoo machine and dipped in ink, which adheres to the tip of the needle. Subsequently, the tattoo machine induces an up-and-down motion of the needle to puncture the top layer of the individual's skin and drive insoluble particles of ink into the dermal layer of skin.

Unfortunately, intricate or large tattoos require a significant amount of time to complete, because the artist must stop tattooing to reapply ink to the needle. This means the needle is removed from the skin when it is dipped. This, in turn, will extend the time for tattooing is dramatically.

The repeated dipping is a disadvantage for both the individual being tattooed and the tattoo artist. While, the tattoo artist would prefer to shorten the time for tattoos to make room for more customers, the customers prefer to decrease the time the experience discomfort while being tattooed. Consequently, what is needed is an apparatus and method that reduces the tattoo time, thereby freeing the tattoo artist to perform more tattoos. Such an apparatus and method would additionally reduce the time that the individual being tattooed would have to sit for the tattoo.

### SUMMARY OF THE INVENTION

An apparatus, and method for using the apparatus, are disclosed which include various aspects not found in the prior art. According to one aspect of the invention, a cylindrical tattoo tube for holding/guiding a reciprocating needle during a tattooing procedure is disclosed. The tattoo needle of the present invention includes a cavity formed in its outer surface for use storing ink to be used during tattooing. In one aspect, the tattoo tube includes tattoo ink thereon which, the tattoo artist may access to provide the ink to the point of a reciprocating needle. The tattoo tube permits freedom of movement of the reciprocating needle, and permits the artist to provide ink to the needle point without the need for the tattoo artist to repeatedly dip the needle in an ink source. In another aspect, the apparatus disclosed includes a means for storing tattoo ink on the tube before tattooing. The means for storing is being done a manner that permits the tattoo artist to provide ink to the needle point manually.

According to the various embodiments of the invention, the cylindrical tattoo tube may include one (or multiple) ink reservoirs for receiving ink to be used for tattooing. The ink reservoir may include an opening for the tattoo artist to include a desired ink therein. The reservoir may further include a channel for transporting the ink from the ink reservoir to a channel exit, wherein the channel exit is located distant from and opposite to the ink reservoir opening, and wherein the channel exit may be proximate to the needle point.

In another aspect of the invention, the apparatus is a cylindrical tattoo tube that allows the tattoo artist to provide multiple different inks to the tattoo site, with each separate ink being included independently of each other. In another aspect, ink may be included in ink capsules, wherein each ink is included in its own capsule. In another aspect of the invention one or more ink capsules may be affixed to the outer surface of the tattoo tube. In yet another aspect, multiple ink capsules may be rotatably affixed to the outer surface of the tattoo tube. According to various aspects of the invention, the tattoo artist may select which of the different ink sources may be used at a given time, by for example, manually rotating the ink sources around the tattoo tube central axis.

### BRIEF DESCRIPTION OF THE DRAWINGS

A more detailed understanding of the invention may be had from the following description, given by way of example, in conjunction with the accompanying drawings wherein:

FIG. 1 shows an exemplary side view of a preferred embodiment of the cylindrical tattoo tube according to various aspects of the present invention;

FIG. 2 shows an exemplary top view of a preferred embodiment of the cylindrical tattoo tube according to various aspects of the present invention;

FIG. 3 shows an exemplary bottom view of a preferred embodiment of the cylindrical tattoo tube according to various aspects of the present invention;

FIG. 4 shows an exemplary side view of a preferred embodiment of the cylindrical tattoo tube according to various aspects of the present invention, including multiple cleaning apertures for cleaning a needlepoint;

FIG. 5 shows an exemplary embodiment of the cylindrical tattoo tube in an exemplary environment as it may be used. In this exemplary embodiment, the cylindrical tattoo tube of the present invention is depicted in a tattoo artist's hand as it may be held during a tattooing procedure;

FIG. 6 is an exemplary method for using a cylindrical tattoo tube according to various aspects of the invention;

FIG. 7 shows an exemplary side prospective view of a preferred embodiment of the cylindrical tattoo tube having multiple liquid reservoirs according to various aspects of the present invention;

FIG. 8 shows an exemplary top view of a preferred embodiment of the cylindrical tattoo tube having multiple liquid reservoirs according to various aspects of the present invention;

FIG. 9 shows an exemplary bottom view of a preferred embodiment of the cylindrical tattoo tube having multiple liquid reservoirs according to various aspects of the present invention;

FIG. 10 depicts an exemplary cylindrical tattoo tube according to the present invention in an exemplary environment in which it may be used. In this exemplary embodiment, the cylindrical tattoo tube of the present invention is depicted being rotated in a tattoo artist hand as it may be rotated during a tattooing procedure

FIG. 11 shows an exemplary side view of another preferred embodiment of a cylindrical tattoo tube according to various embodiments of the invention,

FIG. 12 shows an exemplary top view of a preferred embodiment of a cylindrical tattoo tube according to various embodiments of the present invention;

FIG. 13 shows an exemplary embodiment of a bottom view o f a preferred embodiment of a cylindrical tube according to various embodiments of the present invention;

FIG. 14 depicts a cylindrical tattoo tube of the present invention in an exemplary environment in which it may be used;

FIG. 15 depicts an exemplary embodiment of a cylindrical tattoo tube n in accordance with various embodiments of the present invention, showing adjacent ink reservoirs;

FIG. 16 depicts an exemplary embodiment of an empty ink capsule in accordance with various embodiments of the present invention;

FIG. 17 depicts an exemplary embodiment of an empty ink capsule and plunger in accordance with various embodiments of the present invention;

FIG. 18 depicts an exemplary embodiment of an ink capsule including ink and plunger in accordance with various embodiments of the present invention;

FIG. 19 depicts an exemplary ink capsule placed inside an exemplary cylindrical tattoo tube ink reservoir in accordance with various embodiments of the present invention;

FIG. 20 depicts an exemplary prior art tube that may be used with the present invention;

FIG. 21 depicts ink capsules being affixed to a prior art tube using a tube restraint in accordance with various embodiments of the present invention;

FIG. 22 depicts an exemplary ink capsule restraint useful with various embodiments of the present invention;

FIG. 23 depicts an exemplary ink tube for use with various embodiments of the present invention; and

FIG. 24 depicts another exemplary ink capsule restraint useful with various embodiments of the present invention.

### DETAILED DESCRIPTION OF THE PREFERRED EMBODIMENT

The present invention is described with reference to tattooing, and more particularly, with reference to tattooing with an insoluble ink. However, the invention contemplates using any suitable liquid for tattooing. As such, the terms “ink” and “liquid” may be used interchangeably herein. Further, conventional tattooing procedures are well known and may not be described herein for the sake of brevity. Additionally, the communication of one element to another element, or the communication of one element with another element, as described herein or indicated in the figures, may be physical connections. Further still, the connections may be direct or indirect connections as required to achieve the object and aspects of this invention.

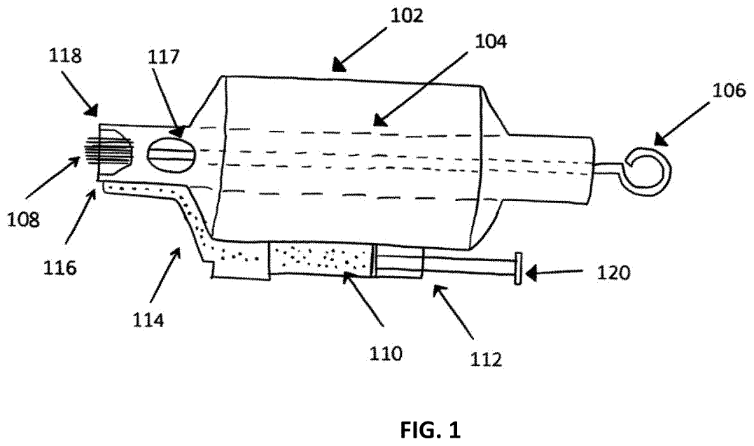

FIG. 1 shows an exemplary side view of a preferred embodiment of a cylindrical tattoo tube 100 according to various aspects of the present invention. As shown, cylindrical tattoo tube 100 includes a cylindrical body 102. Cylindrical body 102 further includes a longitudinal passageway 104 proceeding through cylindrical body 102, wherein longitudinal passageway is proximate to cylindrical body 102 central axis. During use of the invention a conventional tattooing needle 106 may be included through longitudinal passageway 104, such that the needle point 108 of needle 106 protrudes out longitudinal passageway 104 at a longitudinal passageway exit way 118. When needle point 108 protrudes in this manner, the needle point 108 may be placed in contact with an individual's skin during tattooing.

Cylindrical body 102 further includes a cavity 110 (also called an ink reservoir 110, herein) for receiving an ink chosen by the tattoo artist for use in tattooing. Ink reservoir 110 may be distinct from cylindrical body 102. In an alternate embodiment, ink reservoir 110 may be made integral to cylindrical body 102. In yet another exemplary embodiment, ink reservoir 110 may be formed in the outer surface of cylindrical body 102. In still another embodiment, Ink reservoir 110 is in such proximity to cylindrical body 102 that it may be held within a tattoo artists hand when tattooing as described more fully below.

Ink reservoir 110 may include an ink reservoir opening 112 for allowing the ink to be added to, poured in, or injected into ink reservoir 110. In a typical sense, the ink would be added to ink reservoir 110 through ink reservoir open 112 prior to placing the needle point onto the individual's skin.

Ink reservoir 110 may further include a reservoir channel 114 for transporting the ink that has been added to ink reservoir 110 to longitudinal passageway exit way 118. Reservoir channel 114 may be in physical communication with the ink reservoir 110. That is, ink that enters ink reservoir 110 may be transported to longitudinal passageway exit way 118 via reservoir channel 114. More particularly, the ink would be transported from ink reservoir 110 through channel 114 and out reservoir channel exit way 116.

Reservoir channel exit way 116 permits the ink that has been placed in in ink reservoir 110 and transported from the ink reservoir 110 through the reservoir channel 114, to be deposited out reservoir channel exit way 116. In preferred embodiments of the invention, reservoir channel exit way 116 may be in proximity to the longitudinal passageway exit way 118, such that the ink that exits reservoir channel exit way 116 is placed in contact with needle point 108 at longitudinal passageway exit way 118.

A plunger 120 may be included in ink reservoir opening 112 of ink reservoir 110 to facilitate the transport of the ink from ink reservoir 110 through reservoir channel 114 and out reservoir channel exit way 116. As used herein, plunger 120 may be any manually actuated device that may be inserted into reservoir opening 112 for forcing the ink from ink reservoir 110 into reservoir channel 114. In preferred embodiments, plunger 120 operates by displacing the volume of the ink in the ink reservoir 110 with the mass of plunger 120. This action thereby forces the ink into reservoir channel 114 and out reservoir channel exit way 116.

Occasionally, the tattoo artist will need to clean his needle, such as when the artist is changing the color of the ink. The tattoo artist may want to wipe or rinse needle 106 or needle point 108 to remove remnants of a prior used ink. Cylindrical body 102 may include a cleaning aperture 117 (i.e., a tattoo tube wash out 117) along longitudinal passageway 104. In one exemplary embodiment, cleaning aperture 117 is located along longitudinal passageway 104 and in proximity to needle point 108. Aperture 117 provides a tattoo artist access to clean needle 106 and or needle point 108.

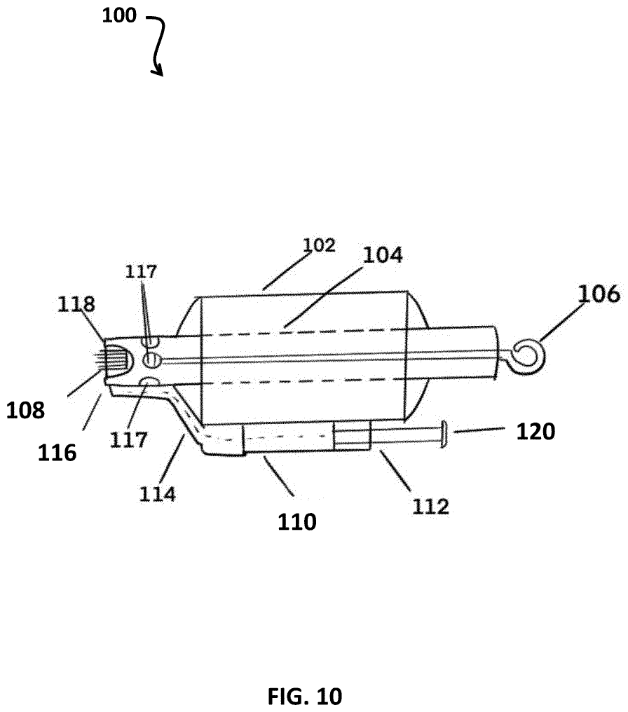

As noted, in some instances, the tattoo artist may desire to clean his needle point 108. To facilitate this, as shown with brief reference to FIG. 10, cylindrical tattoo tube cylindrical body 102 may include multiple cleaning apertures 117 (e.g., wash outs 117) positioned in proximity to needle point 108. Cylindrical body apertures 117 may provide access to needle 106 near needle point 108 to allow the tattoo artist to wipe, clean or rinse the needle and needle point 108.

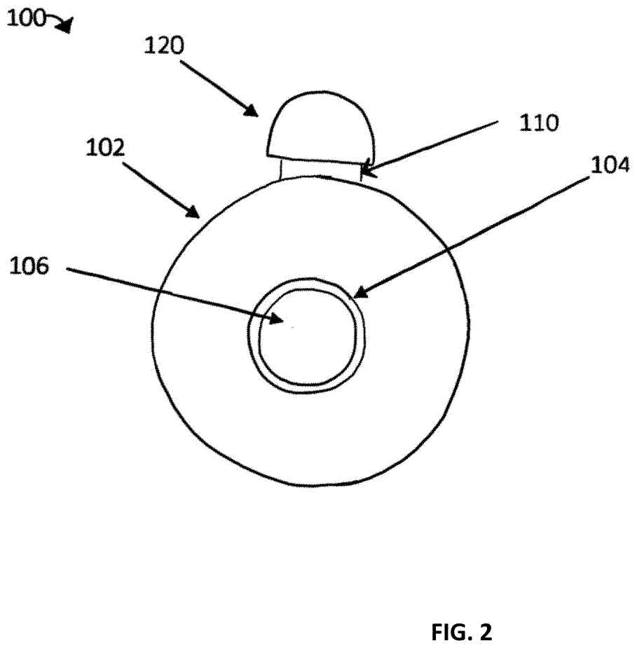

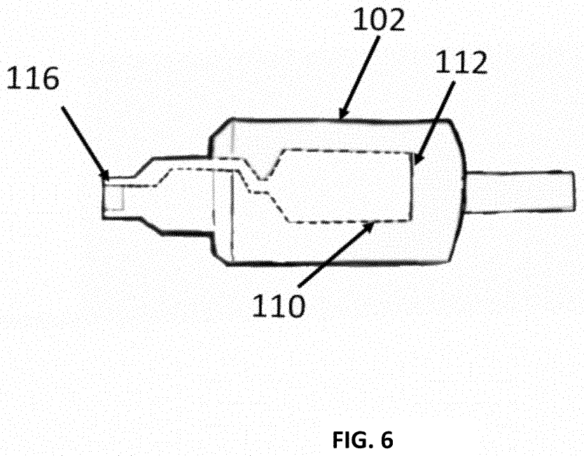

FIG. 2 shows an exemplary top view of a preferred embodiment of cylindrical tattoo tube 100 according to various embodiments of the present invention. As shown, cylindrical body 102 includes longitudinal passageway 104 in which a needle 106 may be inserted. In the embodiment shown, plunger 120 is shown included in reservoir opening 112. Consequently, FIG. 2 depicts the top portion of the plunger 120 positioned alongside cylindrical body 102. In this position, an artist& may manual actuate the plunger 120, thereby dispensing ink into reservoir channel 114 (described more fully with respect to FIGS. 6 and 10). In an exemplary embodiment of the invention, the plunger 120 may be adjacent to cylindrical body 102. In another exemplary embodiment, plunger 120 may be positioned parallel, or substantially parallel with, the longitudinal axis of, cylindrical body 102. In another exemplary embodiment, the orientation of the plunger 120 and the cylindrical body 102 may be such that the plunger 120 and the central axis of cylindrical body 102 form an acute angle.

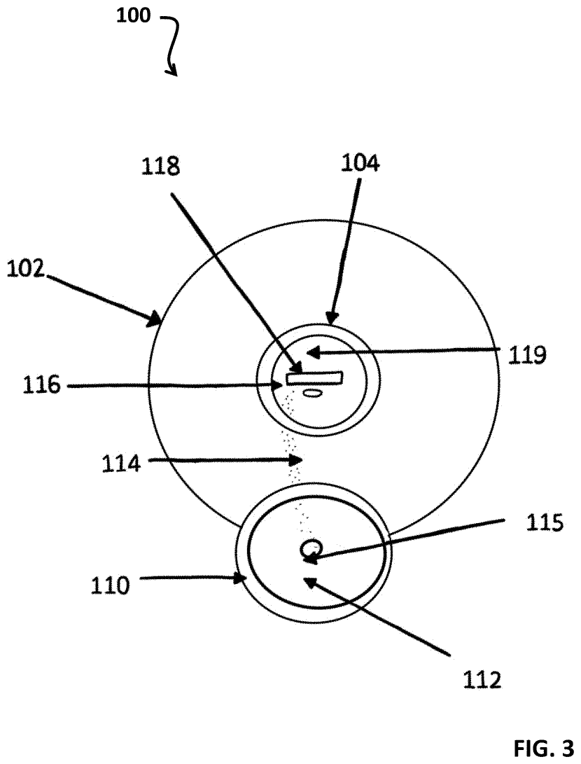

FIG. 3 shows an exemplary top view of a preferred embodiment of cylindrical tattoo tube 100 according to various embodiments of the invention, wherein needle 106 and plunger 120 is depicted removed from cylindrical tattoo tube 100. As shown, ink reservoir 110 (ink cavity 110) includes an opening 112 at a first end of ink reservoir 110 for inserting plunger 120. Ink reservoir 110 further includes a channel exit way 115 positioned substantially opposite ink reservoir opening 112.

Ink exit way 115 may be in further communication with reservoir channel 114. Ink that traverses from ink exit way 115 enters into reservoir channel 114. Reservoir channel 114 may be in communication with channel exit way 116. Ink that traverses from ink exit way 115 into reservoir channel 114 may further exit from channel exit way 116 such that the ink may be placed in proximity to needle point 108. In an alternate embodiment of the invention, the ink that traverses from ink exit way 115 into reservoir channel 114 may further exit from channel exit way 116 such that the ink may be placed in contact with needle point 108.

Cylindrical tattoo tube 100 may further include a longitudinal passageway opening 119 at a first end of longitudinal passageway 104. Needle opening 119 may be formed to receive tattoo needle 106 therein. Cylindrical tattoo tube 100 may further include a longitudinal passageway opening 118 formed at a second end of longitudinal passageway 104. In one exemplary embodiment, longitudinal passageway opening 118 is formed on longitudinal passageway 104 on an opposite end of the passage way to where longitudinal passageway opening 119 is formed. In another exemplary embodiment, the first end of longitudinal passageway 104 is positioned substantially opposite the second end of longitudinal passage way 104. In still another embodiment of cylindrical tattoo tube 100, longitudinal passageway opening 118 is formed adjacent to channel exit way 116. In still another embodiment of cylindrical tattoo tube 102, longitudinal passageway opening 118 is formed substantially adjacent to channel exit way 116.

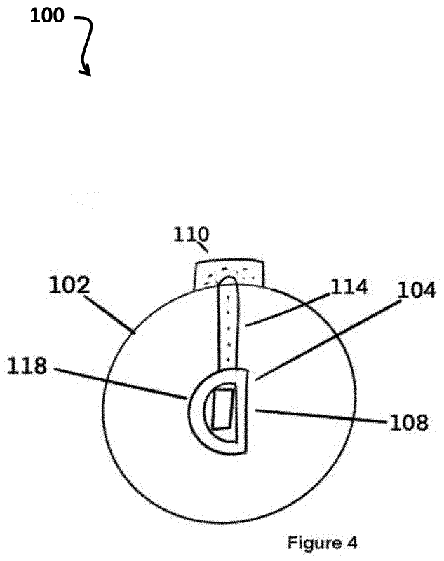

FIG. 4 depicts cylindrical tattoo tube 100 according to various embodiments of the invention, as one would view it from the bottom. As shown, cylindrical tattoo tube body 102 includes ink reservoir 110 positioned adjacent to cylindrical tattoo tube body 102. In another exemplary embodiment, ink reservoir 110 is made integral to cylindrical tattoo tuber body 102.

Cylindrical tattoo tube 100 may further include reservoir channel 114, which is in communication with ink reservoir 110. In another exemplary embodiment, reservoir channel 114 may be in further communication with channel exit way 116. During operation, tattoo ink that is contained in ink reservoir 110 may be provided to reservoir channel 114. The ink provided to reservoir channel 114 may then be provided to channel exit way 116.

FIG. 4 further depicts longitudinal passageway 104 positioned substantially parallel to the cylindrical tattoo tube body 102. Longitudinal passageway 104 may further include longitudinal passageway exit way 118. As shown, needle point 108 may be substantially central to longitudinal passageway exit way 118. In another exemplary embodiment, channel exit way 116 may be positioned in proximity to longitudinal passageway exit way 118.

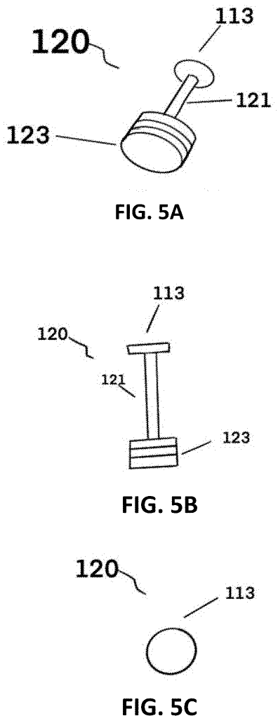

As noted, a plunger 120 may be used with various embodiments of the invention. FIGS. 5A-5C depict a typical plunger 120 that may be used with the present invention, FIG. 54 depicts plunger 120 in perspective view, FIG. 5B depicts plunger 120 in side view, and FIG. 5C depicts plunger 120 from a top view. As shown, plunger 120 may include a plunger flange 113 at a first end. Plunger flange 113 may be formed such that it may be actuated by a human finger. For example, plunger flange 113 may be configured to be pressed by a human thumb. Plunger flange 113 may further include a plunger shaft 121 that is affixed to plunger flange 113. In one exemplary embodiment, plunger shaft 121 may be affixed to the central mass of plunger flange 113. Plunger 120 may further include a plunger head 123 positioned opposite plunger flange 113. Plunger head 123 may be formed in communication with plunger shaft 121. In one exemplary embodiment, the elements of plunger 120 are formed integral to another. In yet another embodiment, the elements of plunger 120 may form a single unitary component.

In one exemplary embodiment plunger 120 may be placed inside ink reservoir 110 for displacing the ink placed by the artist in ink reservoir 110. In another exemplary embodiment, plunger 120 may be inserted in ink reservoir 110 prior to the artist filling ink reservoir 110 with the tattoo ink. In the embodiment shown in FIG. 6, cylindrical tattoo tube body 102 may include ink reservoir 110 this is empty. By empty, what is meant is that ink reservoir 110 does not include tattoo ink therein, or that ink reservoir 110 is substantially free of tattoo ink.

In one exemplary embodiment, plunger 120 may be placed in the empty ink reservoir 110 such that substantially all air is removed from the ink reservoir 110. The air is removed from ink reservoir 110 by placing plunger 120 inside ink reservoir opening 112, and pushing plunger 120 the length of ink reservoir 110 toward channel exit way 116. The tattoo artist may then place reservoir channel exit way 116 in contact with an external ink source (not shown). Ink may then be draw into ink reservoir 110 from the external ink source by slowing drawing plunger 120 back toward in reservoir opening 112 In this case. The ink from the external ink source is drawn through the reservoir channel exit way 116, into reservoir channel 114, and into ink reservoir 110.

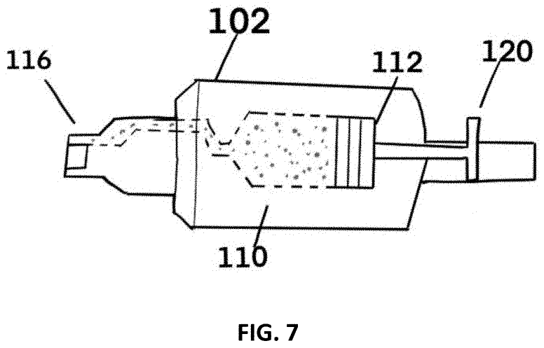

FIG. 7 depicts the result of the action of drawing the ink from the reservoir using the plunger 120, as is described in FIG. 6. As noted, plunger 120 may be drawn toward in reservoir opening 112. However, plunger 120 is not drawn through the reservoir opening 112. In this way, the artist may be able to fill ink reservoir 110 with the ink provided by the external ink source.

FIG. 10 shows an exemplary bottom view of a preferred embodiment of cylindrical tattoo tube 100 according to various aspects of the present invention. As shown, cylindrical body 102 includes longitudinal passageway 104 having longitudinal passageway exit way 118, through which needle point 108 protrudes. Reservoir channel exit way 116 may be placed in proximity to longitudinal passageway exit way 118. Further still, reservoir channel 114 may be in communication with reservoir channel exit way 116 and ink reservoir 110 to facilitate transport of the ink from ink reservoir 114 to reservoir channel exit way 116. As noted, during operation, plunger 120 (shown in FIGS. 1 and 2) may be manual actuation by a tattoo artist to force ink from ink reservoir 110 through reservoir channel 114 and out reservoir channel exit 116.

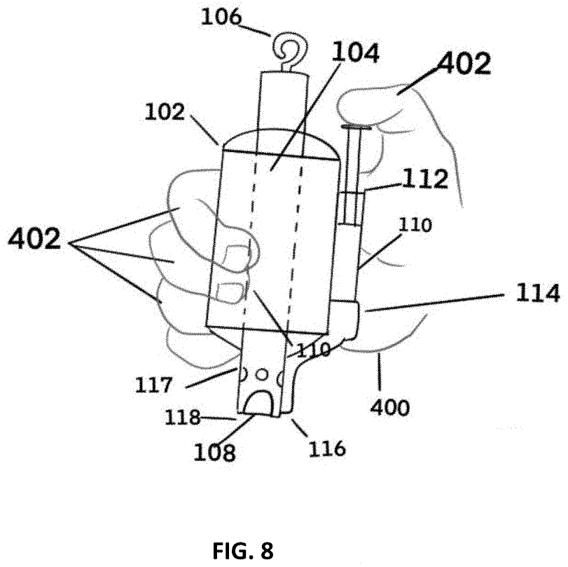

FIG. 8 shows cylindrical tube 100 of present invention in a typical environment in which it may be used. For example, during a typical tattooing session, a tattoo artist may grasp cylindrical tattoo tube 100 by hand having finger 402. More particularly, fingers 402 of the tattoo artist would wrap substantially around cylindrical body 102 to permit the artist to maintain a firm grim during tattooing. The present invention eliminates the need for the tattoo artist to repeatedly dip his tattoo needle in an ink source during the procedure since the ink source for the procedure would be contained in ink reservoir 110. As described below, when the tattoo artist is ready to apply a desired ink to an individual's skin, the ink may be dispensed from the ink reservoir 110 through the reservoir channel 114 and out of the reservoir channel exit way 116 by forcing the plunger 120 into the ink filled ink reservoir 110 using, for example, an artist's thumb 404.

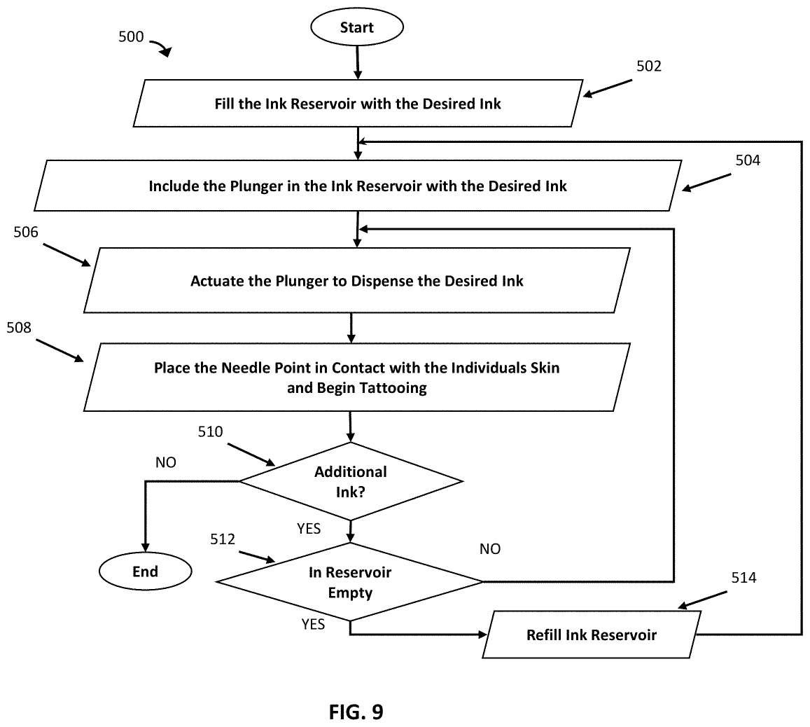

More particularly, FIG. 9 shows an exemplary method 500 of using the cylindrical tattoo tube 100 according to the invention. According to an exemplary method of use, prior to beginning his tattooing procedure, the tattoo artist would fill the ink reservoir 110 through ink reservoir opening 112 with the ink the tattoo artist desires for the procedure (Step 502). After filling the ink in ink reservoir 110, the artist would then include plunger 120 into the ink reservoir opening 112 (Step 504). In a preferred embodiment, the plunger would be placed on the top surface of the ink in the ink reservoir 110.

When the tattoo artist is ready to begin the tattooing procedure, the tattoo artist may transport the ink from the ink reservoir 110, through ink reservoir channel 114 and out reservoir channel exit 116, so that the ink may contact the needle point 108. The tattoo artist facilitates the movement of the ink to needle point 108 by manually actuating the plunger 120 using, for example, the tattoo artist's thumb 404 (or other digit) (Step 506). The tattoo artist may control the amount of the ink dispensed by regulating the amount of manual pressure placed on the plunger 120. Higher downward pressure on plunger 120 may result in a higher volume of ink being transported to exit 116 and contacting needle point 108, while less pressure would result in a lower volume of ink being presented to needle point 108.

Once the tattoo artist has dispensed the desired amount of ink to needle point 108, the tattoo artist may place the needle point 108 in contact with the individual's skin, and begin his tattooing procedure (Step 508). If the tattoo artist needs to use additional ink in his procedure (Step 510), the artist may repeat steps 506 through step 508 until the procedure is ended or the ink in ink reservoir 110 has been substantially or completely used. That is, the tattoo artist may manually press the plunger further into ink reservoir 110 causing the desired amount of ink to come in contact with the needle point 108, as described above. The tattoo artist may thereby continue his tattooing procedure without the need to stop and dip needle point 108 in a separate ink source. The ink source is included with the cylindrical tattoo tube according to various embodiments of the invention. Thus, the tattoo artist can continue his tattooing procedure until the tattooing procedure is completed, or the artist needs to change the ink in ink reservoir 110. In other embodiments described herein, the artist may use this procedure when using a cylindrical tattoo tube with multiple ink reservoirs, which is described more fully below.

In some instances, whether the artist uses a tattoo tube including one or multiple ink reservoirs, the artist may find that his desired ink reservoir is empty (Step 512). In such a case, the artist may refill the reservoir (Step 514), include the plunger and tattoo his subject according to steps 506-510. If the ink reservoir is not empty (Step 512), the tattoo artist may simply actuate the plunger to dispense the desired in (Step 506). The artist may then continue tattooing as described with respect to steps 508-514.

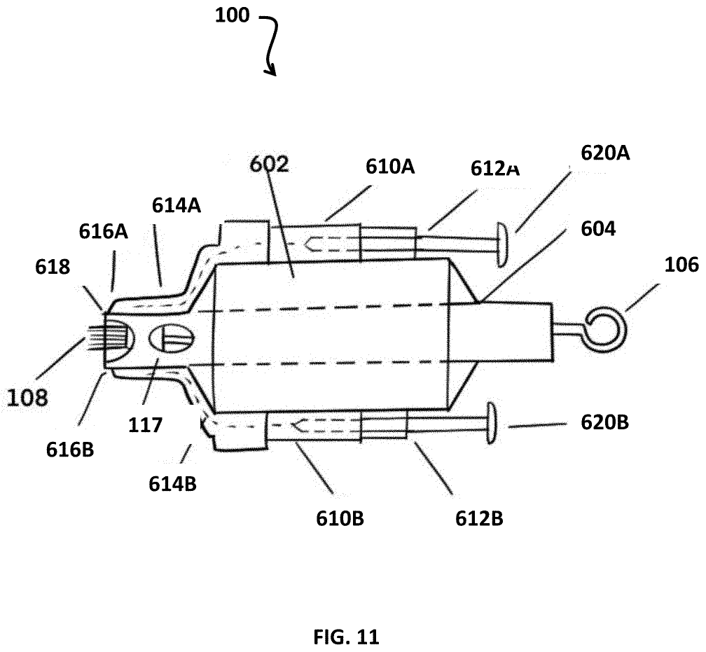

FIG. 11 shows an exemplary side view of another preferred embodiment of a cylindrical tattoo tube 600 according to various aspects of the present invention. As shown, cylindrical tattoo tube 600 includes a cylindrical body 602. Cylindrical body 602 further includes a longitudinal passageway 604 proceeding through cylindrical body 602. The longitudinal passageway 604 may be located proximate to axis of the cylindrical body 602. A conventional tattooing needle 106 may be included through the longitudinal passageway 604, such that the needle point 108 of needle 106 protrudes out a longitudinal passageway exit way 618 of longitudinal passageway 604. A tattoo artist may then be able to use needle point 108 for tattooing an individual's skin in similar manner as was described with cylindrical tattoo tube 100.

Cylindrical body 602 further includes multiple ink reservoirs 610A and 610B for receiving ink for tattooing. In one exemplary embodiment, multiple ink reservoirs 610A and 610B are positioned equal distant from the aperture 117. In another exemplary embodiment, multiple ink reservoirs 610A and 610B are parallel to the cylindrical body 102 central axis. The ink received by reservoir 610A may be the same ink or different ink than is received by reservoir 610B. The ink reservoir 610A may include an ink reservoir opening 612A for allowing a first ink to be added to, poured in, or injected into, the ink reservoir 610A. The ink may be added to ink reservoir 610A through the ink reservoir opening 612A. Similarly, ink reservoir 610B may include an ink reservoir opening 612B for allowing a second ink to be added to, poured in, or injected into, the ink reservoir through the ink reservoir opening 612B. The ink may be added to ink reservoirs through the openings 610A or 610B prior to placing the needle point 108 onto the individual's skin.

Ink reservoir 610A may further include a reservoir channel 614A for transporting the ink that has been added to ink reservoir 610A. Reservoir channel 614A may be in physical communication with reservoir 610A, for receiving the transported ink from ink reservoir 610A. Reservoir channel 614A may further include a reservoir channel exit way 616A that permits the ink that has been placed in reservoir 610A to be transported from ink reservoir 610A and then transported through reservoir channel 614A and out a reservoir channel exit way 616A. In preferred embodiments of the invention, reservoir channel exit way 616A may be in proximity to a longitudinal passageway exit way 618, such that ink that exits the reservoir channel exit way 616A is placed in contact with needle point 108 at longitudinal passageway exit way 618.

Ink reservoir 610B may further include a reservoir channel 614B for transporting the ink that has been added to the ink reservoir 610B. Reservoir channel 614B may be in physical communication with the reservoir 610B, for receiving the transported ink from ink reservoir-610B. Reservoir channel 614B may further include a reservoir channel exit way 616B that permits the ink that has been placed in reservoir 610B to be transported from ink reservoir 610B and then transported through reservoir channel 614B and out a reservoir channel exit way 616B. In preferred embodiments of the invention, reservoir channel exit way 616B may be in proximity to a longitudinal passageway exit way 618, such that ink that exits the reservoir channel exit way 6166 may be placed in contact with needle point 108 at longitudinal passageway exit way 618. In some embodiments, reservoir channel exit way 616A may be located in proximity to reservoir channel exit way 616B. Alternatively, reservoir channel exit way 616A may be located opposite to, or substantially opposite, reservoir channel exit 616B. Even further, reservoir channel exit way 616a may be located adjacent to reservoir channel exit 616B.

A plunger 620A may be included ink inside ink reservoir 610A as is described with respect to plunger 120 and ink reservoir 110 above. Plunger 620A may be useful to facilitate the transport of the ink from reservoir 610A through the reservoir channel 614A and out the reservoir channel exit way 616A. As used herein, a plunger 620A may be a manually actuated device for forcing the ink from reservoir 610A into reservoir channel 614A. In preferred embodiments plunger 620A operates by displacing the volume of the ink in reservoir 610A with the mass of plunger 620A. This action thereby forces the ink into reservoir channel 614A and out the reservoir channel exit way 616A.

Similarly a plunger 620B may be included ink inside ink reservoir 610B as is described with respect to plunger 120 and ink reservoir 110 above. As used herein, plunger 620B may be a manually actuated device for forcing the ink from reservoir 610B into reservoir channel 614B. In preferred embodiments plunger 620B operates by displacing the volume of the ink in reservoir 610b with the mass of plunger 620B. This action thereby forces the ink into reservoir channel 614b and out reservoir channel exit way 616B.

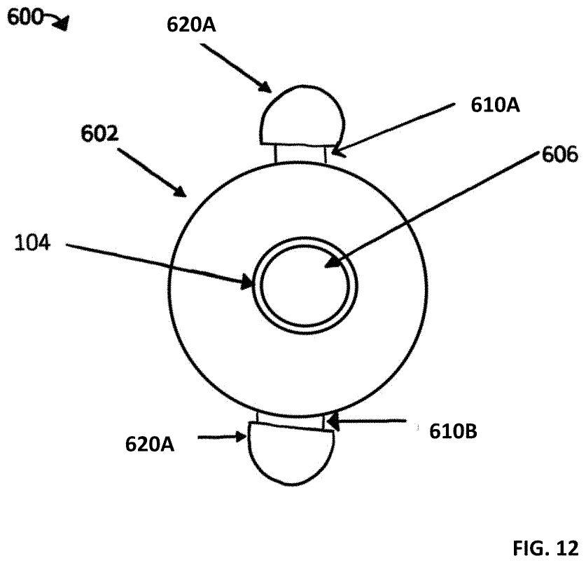

FIG. 12 shows an exemplary top view of a preferred embodiment of cylindrical tattoo tube 600 according to various aspects of the present invention. As shown, cylindrical body 602 includes longitudinal passageway 604 in which needle 606 is inserted. Cylindrical body 602 may include multiple plungers 602A and 602B. In an exemplary embodiment of the invention, plungers 602A and 602B may be located adjacent to cylindrical body 602. In another exemplary embodiment, plungers 620A and 620B may be positioned parallel, or substantially parallel with, the longitudinal axis of cylindrical body 602. In another exemplary embodiment, one of plunger 620A or 620B and the longitudinal axis of cylindrical body 602

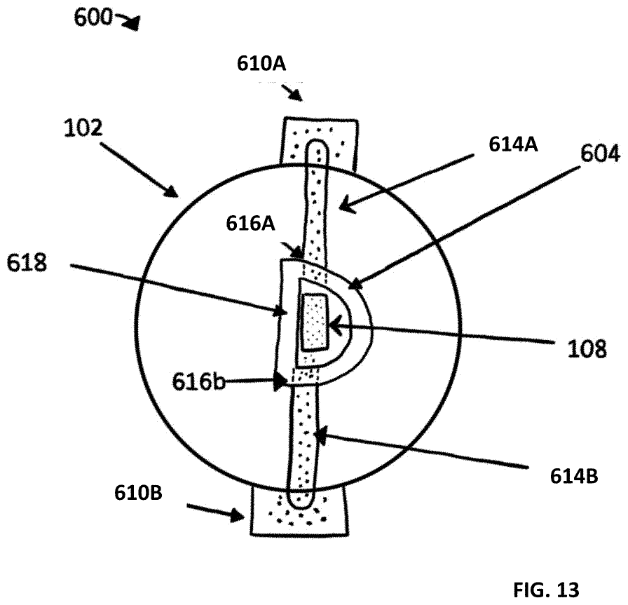

FIG. 13 shows an exemplary bottom view of a preferred embodiment of cylindrical tattoo tube 600 according to various aspects of the present invention. As shown, cylindrical body 602 includes longitudinal passageway 604 having longitudinal passageway exit way 618, through which needle point 108 protrudes. Reservoir channel exit way 616A may be placed in proximity to longitudinal passageway exit way 618. Further still, reservoir channel 614A may be in communication with reservoir channel exit way 616A and ink reservoir 610A. As noted, during operation, plunger 620a may be manual actuation by a tattoo artist to force ink from ink reservoir 610A through reservoir channel 614A and out reservoir channel exit 616A.

Reservoir channel exit way 616B may be placed in proximity to longitudinal passageway exit way 618. Further still, reservoir channel 614b may be in communication with reservoir channel exit way 616B and ink reservoir 610B. As noted, during operation, plunger 620B may be manual actuation by a tattoo artist to force ink from ink reservoir 610b through reservoir channel 614B and out reservoir channel exit 616B.

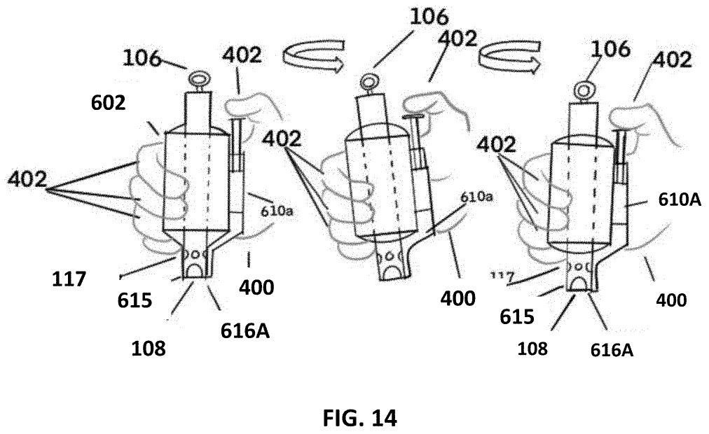

FIG. 14 shows cylindrical tattoo tube 600 of the present invention in a typical environment in which it may be used. For example, during a typical tattooing session, a tattoo artist would grasp cylindrical tattoo tube 600 by hand. More particularly, fingers 402 of the tattoo artist's hand 400 would wrap substantially around cylindrical body 602 to permit the artist to maintain a firm grip of cylindrical tattoo tube 600 during tattooing.

The present invention eliminates the need for the tattoo artist to repeatedly dip his tattoo needle point in his ink source during the procedure since the ink for the procedure would be contained in ink reservoirs 610A and 610B. Additionally, when the cylindrical tattoo tube of present invention includes multiple ink reservoirs, the present invention eliminates the need to exchange a first ink that is in an ink reservoir, for a second ink that the artists may wish to use. When the tattoo artist is ready to apply a second desired ink to an individual's skin, the artist need only rotate the cylindrical tattoo tube so that the tattoo artist's thumb may actuate the plunger corresponding to the desired ink.

For example, a tattoo artist may begin tattooing using a first ink in ink reservoir 610A. The first ink may be dispensed from the ink reservoir 610A through the reservoir channel 614A and out of the reservoir channel exit way 616A by forcing the plunger 620A into the ink filled ink reservoir 610A using an artist's thumb 404. When the artist wishes to use a second ink in ink reservoir 610B, the artist may rotate cylindrical tattoo tube 600 to enable the artist's thumb to actuate plunger 620b. In that way, the desired ink may be dispensed from the ink reservoir 610B through reservoir channel 614b and out of reservoir channel exit way 616B by forcing the plunger 620B into the ink filled ink reservoir 610B again using an artist's thumb 404. In all other respects, the way in which cylindrical tattoo tube 600 is used in tattooing is similar in manner as was described with respect to the use of cylindrical tattoo tube 100 with respect to FIG. 9.

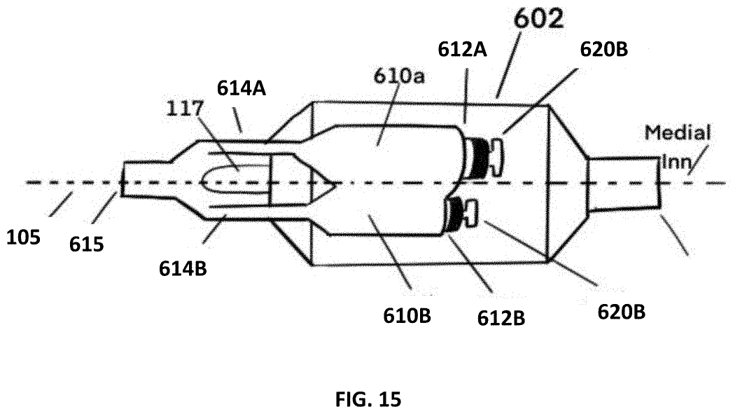

As previously noted, in one exemplary embodiment, multiple ink reservoirs 610A and 610B are positioned equal distant one from the other. As shown in FIG. 15, ink reservoirs 610A and 610B may be positioned on cylindrical body 602, wherein ink reservoir 610A is substantially adjacent to the ink reservoir 610B along cylindrical body 602 medial line, where the medial line is drawn co-axially with the longitudinal axis of cylindrical body 602. In one exemplary embodiment, ink reservoir 610A may be comprised of a different volume than ink reservoir 610B. That is, ink reservoir 610A by be more voluminous than ink reservoir 610B.

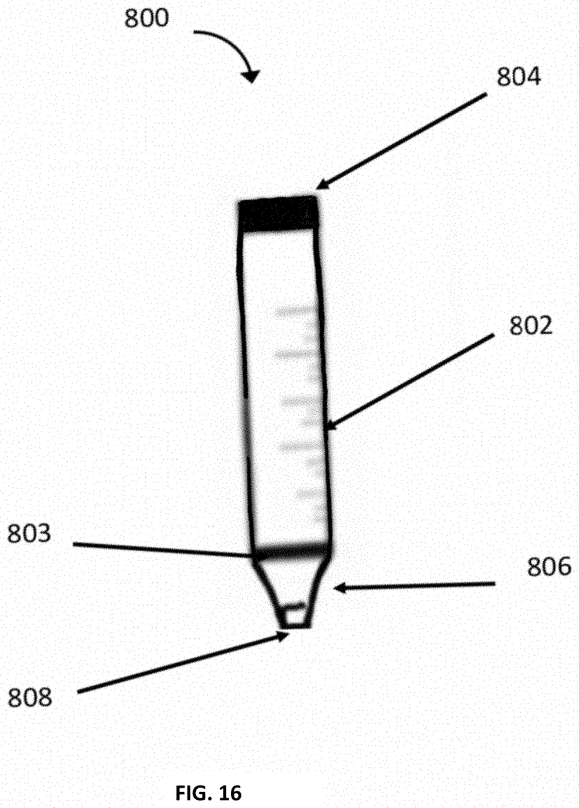

In another exemplary embodiment of the invention, the tattoo artist may provide tattoo ink to a tattoo needle using an ink capsule containing a desired tattoo ink. FIGS. 16-19 depict an exemplary ink capsule 800 used according to the present invention. As shown in FIG. 16, ink capsule 800 may include a hallow cylindrical body 802. Capsule cylindrical body 802 may include a capsule opening 804 at a first end. Capsule cylindrical body 802 may further include a tapered end 806 on a capsule cylindrical body second end 803. The widest end of the tapered end 806 is in communication with the cylindrical body second end 803. The narrowest end of the tapered end 806 may include a tapered end opening 808. Capsule 800 may further include a plunger 120 of similar construction and operation as is described with respect to FIG. 5.

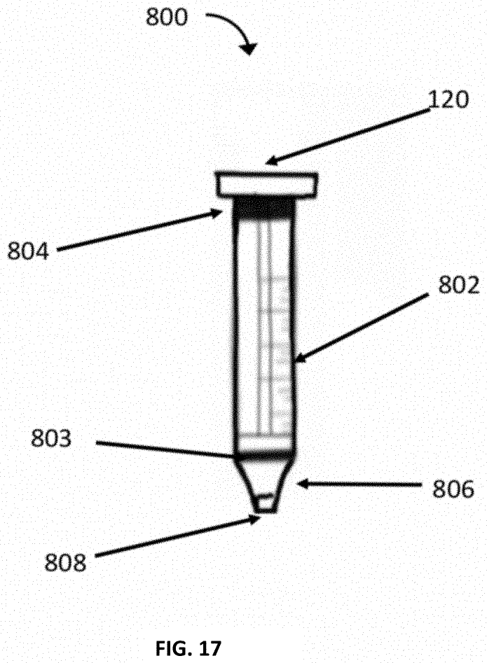

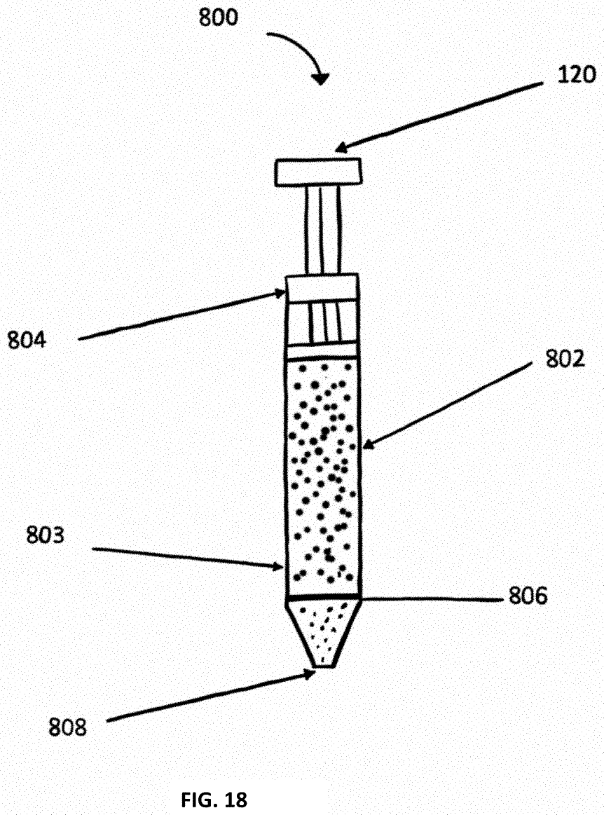

As shown in FIG.17, plunger 120 may be inserted in capsule opening 804 and pushed the length of capsule cylindrical body 802 from capsule opening 804 to capsule tapered end 806. In this way, air that existed in capsule cylindrical body 802 prior to insertion of plunger 120 is substantially pushed out tapered end opening 808. Tapered end opening 808 may be placed in communication with an ink source (not shown) for providing tattoo ink to capsule cylindrical body. For example, FIG. 18 depicts ink capsule 800, wherein capsule tapered end 806 has been placed in contact with an external ink source and plunger 120 has been drawn the length of capsule 800 from tapered end 806 toward capsule opening 804. In the depiction shown, plunger 120 has been pulled from its position in the proximity of tapered end 806 to capsule opening 804 such that ink is drawn into capsule body 802.

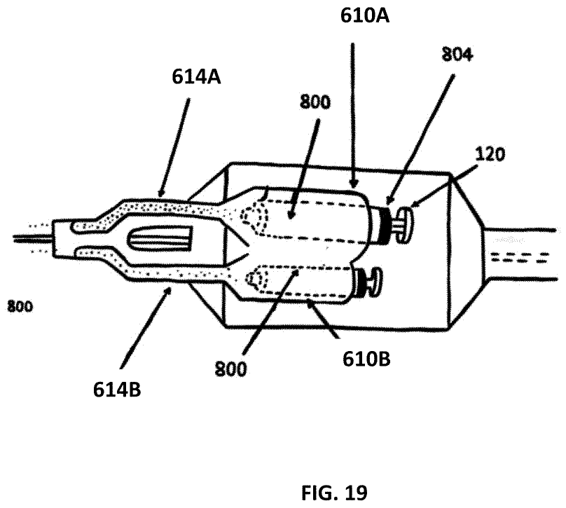

In one exemplary embodiment, as shown in FIG. 19, ink capsule 800 may be configured to be placed in or positioned inside any of the ink cavity or ink reservoirs described herein. For example, with reference to cylindrical tattoo tube 600, ink capsule 800 may be configured to fit within ink reservoir 610a, or ink reservoir 610b, depending on which embodiment is in use.

Although the description is exemplary of placing ink capsule 800 into ink reservoir 610a and 610b, the description applies to the instance where ink capsule 800 is inserted into ink reservoir 110. That is, where ink capsule 800 is configured to be placed or positioned within ink reservoir 610a, ink capsule 800 tapered end opening 808 may be configured to communicate with reservoir channel 614a. In such instance, ink that is in ink reservoir channel 614a may be provided to ink capsule 800 via tapered end opening 808 by, for example, pulling plunger 120 from tapered end 806 toward ink capsule opening 804.

In this way, the suction created when the plunger is pulled draws the ink from ink reservoir channel 614a, through ink capsule opening 804 into the tapered end 806 and further into capsule cylindrical body 802. Similarly, ink that is in ink capsule 800 may be provided to reservoir channel 614a by, for example, pushing plunger 120 from a position at ink capsule opening 804 toward tapered end 806. In this way, ink that is in capsule cylindrical body 802 in forced into tapered end 806. The pushing action of the plunger 120 may further push the ink from tapered end 806 through tapered end opening 808 and into reservoir channel 614a.

Similarly, as shown in FIG. 19, ink capsule 800 may be configured to be placed in or positioned within ink reservoir 610B, ink capsule 800 tapered end opening 808 may be configured to communicate with reservoir channel 614B. In such instance, ink that is in ink reservoir channel 614B may be provided to ink capsule 800 via tapered end opening 808 by, for example, pulling plunger 120 from tapered end 806 toward ink capsule opening 804. In this way, the suction created when the plunger is pulled draws the ink from ink reservoir channel 614A, through ink capsule opening 804 into the tapered end 806 and further into capsule cylindrical body 802. Similarly, ink that is in ink capsule 800 may be provided to reservoir channel 614B by, for example, pushing plunger 120 from a position at ink capsule opening 804 toward tapered end 806. In this way, ink that is in capsule cylindrical body 802 in forced into tapered end 806. The pushing action of the plunger 120 may further push the ink from tapered end 806 through tapered end opening 808 and into reservoir channel 614B.

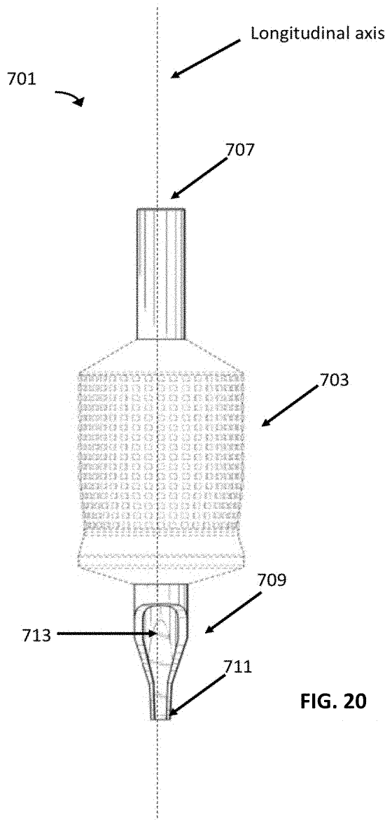

In yet another exemplary embodiment, ink capsule 800 may be used in conjunction with a prior art tube 701, for example, such as is depicted in FIG. 20. In this instance, ink capsule 800 may be affixed to the exterior surface of prior art tube 701. In one exemplary embodiment ink capsule 800 may be removably affixed to the exterior surface of prior art tube 701.

FIG. 20 depicts an exemplary prior art tattoo tube 701 that may be used with the present invention. As shown, prior art tattoo tube 700 may include a hollow cylindrical body 703 and a first prior art tube open end 707 for inserting a tattoo needle. Prior art tattoo tube 701 may include a prior art tube tapered end 709 positioned on the prior art tube 701 lateral axis. Prior art tube tapered end 709 may be positioned on the opposite end of prior art tube cylindrical body 703. Prior art tube tapered end 709 may further include a prior art tube tapered end opening 711 configured such that a tattoo needle inserted in prior art tube opening 707 may protrude through prior art tube tapered end opening 711. Prior art tube tapered end 709 may further include a prior art tube washout 713 for providing access to a needle protruding through prior art tube tapered end opening 711.

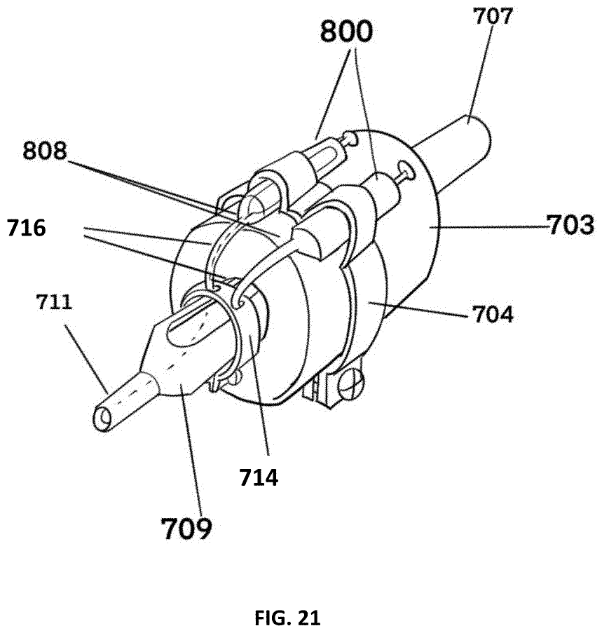

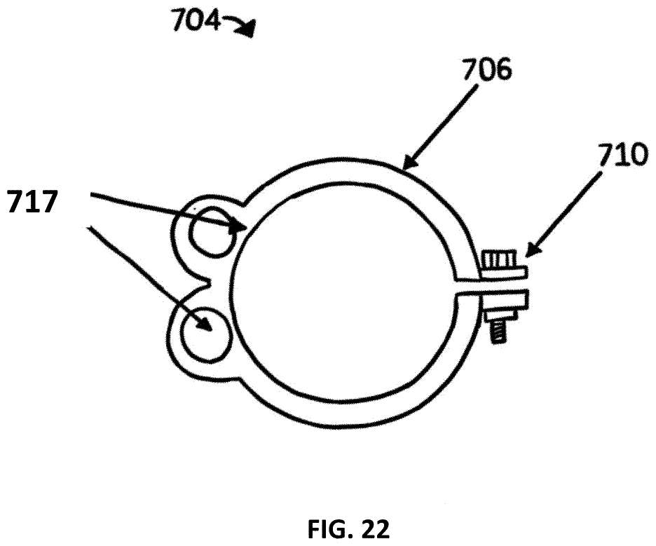

As noted, ink capsules 800 may be affixed to the exterior surface of a prior art tube cylindrical body 703. In one exemplary embodiment shown in FIG. 21, ink capsules 800 may be affixed to prior art tube cylindrical body 703 using an ink capsule restraint 704. With brief reference to FIG. 22, ink capsule restraint 704 suitable for use with the present invention is depicted prior art tube cylindrical body 703.

Ink capsule restraint 704 may be shaped to substantially encircle prior art tube cylindrical body 703. That is, ink capsule restraint 704 may include capsule holding portions 707 717, which are configured to the outer surface of capsule 800. In one exemplary embodiment, ink capsules 800 may be oval in shape. In such an instance, ink capsule 800 may include a capsule opening of similar construction as capsule opening 808. Capsule restraint 704 may comprise a strap 706 for holding in capsules 800 in physical abutment with cylindrical body 703. In one exemplary embodiment, capsule restraint 704 may be held securely to the outer surface of prior art tube cylindrical body 703 using a fastener 710.

Strap 706 may be comprised of metal, plastic, rubber or other material suitable for affixing capsules 800 to cylindrical body 703. As shown, capsule restraint 704 may include a restraint fastener 710 for ensuring that ink capsules 800 remain removably affixed to the prior out tube cylindrical body 703 outer surface. In one exemplary embodiment, strap 706 is elongated. As such, strap 706 may be used to encircle ink capsules 800 against the outer surface of cylindrical tube body 703. Strap 706 maintains ink capsules 800 in abutment with cylindrical tube body using restraint fastener 710. That is, a first end of elongated strap 706 may be fastened to a second end of elongated strap 706 using a suitable fastener 710, such as, as screw, rivet, snap or the like.

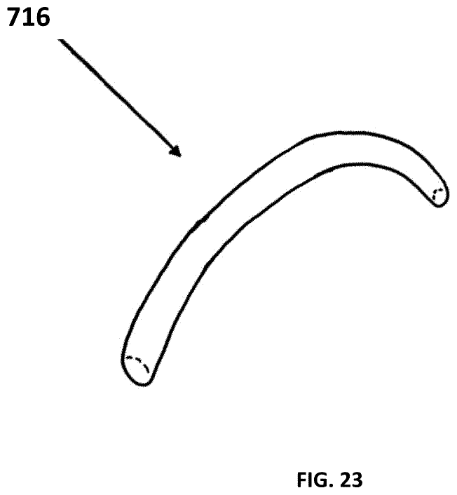

With reference again to FIG. 19, ink capsule opening 808 may be configured to communicate with prior art tube wash out 713 using an ink tube 716 (shown in FIG. 23). Ink tube 706, for receiving ink contained in ink capsule 800. In an alternate embodiment, ink tube 716 may be configured to communicate with ink capsule opening 808 for providing ink to ink capsule 800 via ink capsule opening 808. Ink tube 716 may be comprised of any suitable material for transporting liquid, such as, ink. Ink tube 716 may be plastic, rubber or the like. In one exemplary embodiment, ink tube 716 may be made integral to ink capsule 800.

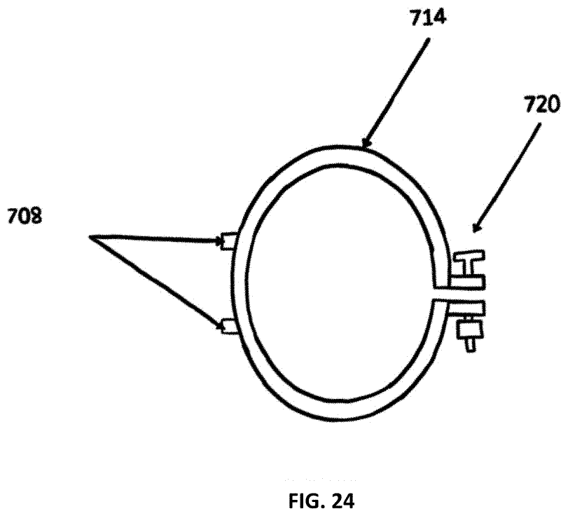

As noted, ink tube 716 may be in further communication with prior art tube washout 713. In one exemplary embodiment, ink tube 716 may be in communication with prior art tube washout 713 for providing ink therein. In yet another exemplary embodiment, ink tube 716 may be in communication with an ink tube band 714 positioned in proximity to prior art tube washout 713. With reference to FIG. 24, Ink band 714 may be constructed of similar material as was described above with respect to strap 706. The ink tube band 714 may encircle prior art tube tapered end 709. In another exemplary embodiment, ink tube band 714 may include a fastener 720 for ensuring ink tube band 714 remains removably affixed to prior art tube tapered end 709. Fastener 720 may be of similar construction as fastener 710. In yet another exemplary embodiment, an ink tube band fastener 720 for ensuring that ink tube band 714 remains removable affixed to prior art tapering end 709.

Ink tube band 714 may include projections 708 for accepting one end of the end of the ink tube 716 positioned opposite ink capsule opening 808. In one exemplary embodiment, projections 708 may be configured such that ink tube 716 may be affixed thereto. In addition, projections 708 may include an ink band canal formed through ink tube band 714 for permitting ink that is contained in ink tube 716 to flow freely from ink tube 716 to prior art tube washout 713. In other exemplary embodiment, ink tube 706 may be of sufficient length such that it is positioned through the washout 713 such that the ink tube 716 provides ink to prior art tube tapered end opening 711.

With return reference to FIG. 21, ink capsule 800 is shown in communication with ink tube 706. As shown, tapered end 806 includes tapered end opening 808 which is further connected to ink tube 706. With reference to FIG. 24, what is shown is ink tube 716 connected to ink tube band 714. Further, what is shown is that ink tube 716 may be affixed to projections 708 for providing ink to washout 713 via ink band aperture 705.

It should be noted that various embodiments of the invention are described with respect to the individual elements of the invention being formed of one piece. However, it is contemplated that the present invention may have elements that are detachable, such as, the plunger or ink reservoir. Further still, such modifications are within the spirit and scope of the invention.

1. A cylindrical tattoo tube for holding a reciprocating needle during tattooing, the tattoo tube comprising
a. a substantially cylindrical body, having
i. an outer surface including a first cavity formed therein;
ii. an inner passageway formed central to the cylindrical body, the inner passageway formed completely through the cylindrical body, the inner passageway formed substantially parallel to the cylindrical body central axis; and
iii. a wash out formed substantially at a first end of the inner passageway.

2. A tattoo tube according to claim 1, having a first plunger, wherein the first plunger is constructed to be included within the first cavity.

3. A tattoo tube according to claim 2, further including a second cavity formed in the outer surface; and a second plunger wherein the second plunger is constructed to be included within the second cavity.

4. A tattoo tube according to claim 3, wherein the first cavity is formed adjacent to the second cavity.

5. A tattoo tube according to claim 1, having a hollow ink capsule configured to be included within the first cavity, wherein the hollow ink capsule has a first open end and a second open end, wherein the hollow ink capsule first open end is configured to receive tattoo ink therein.

6. A tattoo tube according to claim 5, further including a first plunger, wherein the first plunger is configured to be included within the hollow cavity first open end.

7. A system for providing tattoo ink to a tattoo tube during tattooing including:
a. a first restraining strap encircling an outer surface of the tattoo tube, wherein the first restraining strap is affixed to the outer surface of the tattoo tube;
b. a first ink capsule in abutment with the outer surface of the tattoo tube, the first ink capsule in abutment with the first restraining strap, wherein the first restraining strap holds the first ink capsule in abutment with the outer surface of the tattoo tube.

8. A system according to claim 7, wherein the first ink capsule includes an ink capsule first open end and an ink capsule second open end, wherein the ink capsule first open end is configured to permit tattoo ink to pass therethrough.

9. A system according to claim 8, further comprising an ink tube having a ink tube first open end and an ink tube second open end, wherein the ink tube first open end is in communication with the ink capsule first open end, wherein the ink tube is configured to transport the tattoo ink.

10. A system according to claim 9, further comprising an ink tube band in communication with the ink tube second open end, wherein the ink tube band is in configured to affix the ink tube second open end to the tattoo tube outer surface.

11. A system according to claim 10, where the ink tube band in proximity to a tattoo tube washout.

12. A system according to claim 8, further comprising a first plunger, wherein the first plunger is constructed to be included in the ink capsule first open end.

13. A tattoo tube comprising:
a. an elongated tubular body, comprising,
i. a tattoo tube outer surface;
ii. a tattoo tube inner passageway formed central to the tattoo tube elongated tubular body, the inner passageway formed substantially parallel to the tattoo tube elongated tubular body central axis, wherein the tattoo tube inner passageway is formed completely through the tattoo tube elongated tubular body; and
iii. a tattoo tube wash out formed in the tattoo tube outer surface, wherein the tattoo tube wash out is formed at a first end of the tattoo tube inner passageway;
b. a first ink reservoir formed adjacent to the tattoo tube outer surface, the first ink reservoir having a first ink reservoir first open end.

14. A tattoo tube according to claim 13, having a first plunger, wherein the first plunger is constructed to be included within the first ink reservoir first open end.

15. A tattoo tube according to claim 14, further including:
a. a second ink reservoir formed adjacent to the tattoo tube outer surface, wherein the second ink reservoir includes a second ink reservoir first open end; and
b. a second plunger wherein the second plunger is constructed to be included within the second ink reservoir first open end.

16. A tattoo tube according to claim 15, wherein the first ink reservoir is formed adjacent to the second ink reservoir.

17. A tattoo tube according to claim 13, having a hollow ink capsule configured to be included within the first ink reservoir, wherein the hollow ink capsule has a first open end and a second open end, wherein the hollow ink capsule first open end is configured to receive tattoo ink therein.

18. A tattoo tube according to claim 17, further including a first plunger, wherein the first plunger is configured to be included within the hollow ink capsule first open end.

19. A tattoo tube according to claim 18, further including an ink transport channel having a transport channel first open end, and an ink transport channel second open end, wherein the ink transport channel first open end is in communication with the hollow ink capsule second open end.

20. A tattoo tube according to claim 19, wherein the ink transport channel second open end is in proximity to the tattoo tube wash out.
